(19) United States
(12) Patent Application Publication
(10) Pub. No.: US 2023/0225977 A1
(43) Pub. Date: Jul. 20, 2023

(54) FORMULATION COMPRISING DAPRODUSTAT

(71) Applicant: GLAXOSMITHKLINE INTELLECTUAL PROPERTY (NO.2) LIMITED, Middlesex (GB)

(72) Inventors: Sayantan CHATTORAJ, Collegeville, PA (US); Scott Ryan CROY, Collegeville, PA (US); James Anderson MCLEOD, Collegeville, PA (US)

(21) Appl. No.: 18/010,245

(22) PCT Filed: Jun. 17, 2021
(86) PCT No.: PCT/EP2021/066386

§ 371 Date: Dec. 14, 2022

Related U.S. Application Data
(60) Provisional application No. 63/041,403, filed on Jun. 19, 2020.
(60) Provisional application No. 63/043,292, filed on Jun. 24, 2020.

(51) Int. Cl. A61K 9/20; A61K 31/515; A61K 45/06

(52) U.S. Cl. CPC A61K 9/2077; A61K 31/515; A61K 9/2054; A61K 9/2009; A61K 9/2018; A61K 45/06

(57) ABSTRACT

The present disclosure relates to an immediate release tablet of daprodustat having good tensile strength. In other aspects, medical uses of the immediate release tablet and dosage regimens for using the immediate release tablet are disclosed.

### FIELD OF THE INVENTION

The present disclosure relates to an immediate release tablet of daprodustat having good tensile strength. In other aspects, medical uses of the immediate release tablet and dosage regimens for using the immediate release tablet are disclosed.

### BACKGROUND TO THE INVENTION

Tablets must possess suitable mechanical strength to avoid crumbling or breaking during downstream processing and when handling, whilst ensuring suitable disintegration after oral administration. Accordingly, assessment of mechanical strength of tablets is routinely performed during tablet manufacturing as a measure of product robustness.

The physical properties of the drug substance and excipients used in a tablet strongly influence the mechanical characteristics of the obtained tablets, including tablet tensile strength. For example, Shah and colleagues reported the impact of bulk density of two widely used microcrystalline cellulose preparations on tablet tensile strength and friability (World Journal of Pharmaceutical Research, 2017, 6(10):841-852). The authors reported a correlation between bulk density and tensile strength, with the parameters being inversely proportional to each other. Similarly, a correlation was found between bulk density and friability with these parameters also being inversely proportional to one another.

Daprodustat is a prolyl hydroxylase inhibitor that is currently in development for the treatment of anaemia due to chronic kidney disease. A tablet formulation of daprodustat is disclosed in WO2019/052133. Daprodustat tablets having tablet tensile strengths sufficient to permit normal storage, distribution and handling are needed.

### SUMMARY OF THE INVENTION

In a first aspect, the invention provides an immediate release tablet comprising from 1 to 10 mg (measured as the free acid) daprodustat or a pharmaceutically acceptable salt thereof which has a tablet tensile strength of greater than or equal to 1.7 MPa following compaction of the tablet core at a pressure in the range of 200 to 290 MPa.

In a second aspect, the invention provides the immediate release tablet of the invention for use in therapy, including in the treatment of anemia due to chronic kidney disease.

In a third aspect, the invention provides the immediate release tablet of the invention for use in the treatment of anemia due to chronic kidney disease, wherein the immediate release tablet of the invention is administered once daily at one of the following doses: 1, 2, 4, 6, 8, 10, 12, 16 and 24 mg (dose of free acid) in accordance with the following dosage regimen:
a. where the haemoglobin concentration is ≥12 g/dL, daprodustat therapy is ceased until the haemoglobin concentration is <11.5 g/dL and therapy is commenced at one dose step lower;
b. where the haemoglobin concentration is in the range ≥9.5 to <11.5 g/dL, the dose is maintained;
c. where the haemoglobin concentration is in the range >11 to ≥11.5 g/dL at two consecutive clinic visits and there has been an increase or no change in the haemoglobin concentration since the last visit, the dose is reduced by one dose step;
d. where the haemoglobin concentration is in the range >11.5 to <12 g/dL and there has been a decrease in haemoglobin concentration since the last visit, the dose is maintained.
e. where the haemoglobin concentration is in the range >11.5 to <12 g/dL and there has been an increase or no change in the haemoglobin concentration since the last visit, the dose is reduced by one dose step;
f. where the haemoglobin concentration is in the range ≥9.5 to <10 at two consecutive clinic visits and there has been a decrease or no change in the haemoglobin concentration since the last visit, the dose is increased by one dose step;
g. where the haemoglobin concentration is in the range 7.5 to <9.5 g/dL and there has been an increase in haemoglobin concentration of ≥0.5 g/dL since the last visit, the dose is maintained;
h. where the haemoglobin concentration is in the range 7.5 to <9.5 g/dL and there has been a decrease, no change or an increase of <0.5 g/dL in haemoglobin concentration since the last visit, the dose is increased by one dose step;
i. where the haemoglobin concentration is <7.5 g/dL, the dose is increased by one dose step;
j. where there has been an increase in haemoglobin concentration of >2 g/dL over 4 weeks, or an increase in haemoglobin concentration of >1 g/dL over 2 weeks, the dose is reduced by one dose step; and
k. where there has been a decrease in haemoglobin concentration of >2 g/dL over 4 weeks, or a decrease in haemoglobin concentration of >1 g/dL over 2 weeks, the dose is increased by one dose step.

In one embodiment, the immediate release tablet of the invention is administered once daily at one of the following doses: 1, 2, 4, 6, 8, 12, 16 and 24 mg (dose of free acid) in accordance with the dosage regimen described herein.

### DESCRIPTION OF DRAWINGS/FIGURES

FIG. 1 is a Scanning Electron Microscopy image of non-solvated crystalline form of daprodustat free acid.

FIG. 2 shows an X-ray powder diffraction pattern of a non-solvated crystalline form of daprodustat free acid.

### DETAILED DESCRIPTION OF THE INVENTION

### Definitions

An immediate release tablet comprising from 1 to 10 mg (measured as the free acid) daprodustat or a pharmaceutically acceptable salt thereof is a tablet comprising from 1 to 10 mg (measured as the free acid) daprodustat or a pharmaceutically acceptable salt thereof that meets the following dissolution criteria:
1. A mean (based on at least 12 tablets) of 85% or more of the daprodustat thereof contained in the tablet dissolves within 45 minutes or less using United States Pharmacopeia (USP) Apparatus 2 with a rotational speed of 50±2 rpm and a dissolution volume of 500±5 mL for tablets containing <2 mg daprodustat (measured as the free acid) and 900±9 mL for tablets containing mg daprodustat (measured as the free acid) in a pH 6.8 buffer or Simulated Intestinal Fluid USP without enzymes.

In one embodiment, the dissolution profile of an immediate release tablet comprising from 1 to 10 mg (measured as the free acid) daprodustat or a pharmaceutically acceptable salt thereof using United States Pharmacopeia (USP) Apparatus 2 under the conditions specified above must additionally exhibit an f2 value ≥50 compared to a tablet as described in Example 3 containing the same dose of active pharmaceutical ingredient. In one embodiment, the tablet of Example 3 was compacted using a main compaction pressure of 200-290 MPa, more particularly 240-260 MPa and even more particularly, about 250 MPa.

In the context of the invention, the term tablet core refers to the entire tablet excluding any coating.

Tablet tensile strength is a measure of tablet robustness and may be measured by any method known in the art, for example by use of a diametral compression test. In one embodiment, tablet tensile strength (measured in MPa) is determined using the method published by Pitt and Newton (Journal of Materials Science 23, 2723-2728, 1988.).

The term glidant refers to an excipient capable of improving the flow of a poorly flowing powder. Glidants include colloidal silicon dioxide, talc, starch and magnesium stearate. Whilst magnesium stearate is frequently used as a lubricant, it also increases flow of poorly flowing powders at concentrations typically used in formulations (improvements in the flow of spray-dried lactose have been reported with the addition of up to 4% magnesium stearate; see Morin and Brians, AAPS Pharm Sci Tech. 2013 September; 14(3): 1158-1168).

In the context of the invention, a compartment of the tablet is a portion of the tablet that has the same composition. For example, granules and extragranular spaces may be separate compartments within a tablet. Different layers in a multilayer tablet could form separate compartments. Additionally, the entire monolithic tablet can be a single compartment.

Active Pharmaceutical Ingredient

The tablet of the invention comprises between 1 and 10 mg (measured as the free acid) daprodustat or a pharmaceutically acceptable salt thereof. Daprodustat is the USAN, INN and JAN name for the compound N-[(1,3-dicyclohexyl-6-hydroxy-2,4-dioxo-1,2,3,4-tetrahydro-5-pyrimidinyl)carbony]glycine (the IUPAC name for this compound is N-[(1,3-Dicyclohexylhexahydro-2,4,6-trioxopyrimidin-5-yl)carbonyl]glycine). Daprodustat exhibits keto/enol tautomerism. All tautomers of daprodustat, including mixtures thereof, are intended to be encompassed within the scope of the invention.

Daprodustat or pharmaceutically acceptable salts thereof may be prepared in accordance with the process disclosed in WO2007/150011. In one embodiment, the tablet contains between 1 and 10 mg daprodustat free acid.

In a particular embodiment, the daprodustat free acid is a non-solvated crystalline form characterised by:
1) a sharp melting point from 240-242° C. as measured by thermogravimetric analysis; and/or
2) an X-ray powder diffraction (XRPD) pattern comprising at least five diffraction angles, when measured using Cu K_{α} radiation, selected from the group consisting of 4.0+/−0.2, 6.4+/−0.2, 7.5+/−0.2, 8.0+/−0.2, 15.2+/−0.2, 17.2+/−0.2, 18.6+/−0.2, 19.3+/−0.2, 19.9+/−0.2, 20.4+/−0.2, 21.0+/−0.2 and 24.1+/−0.2 degrees 2θ.

This crystalline form may be prepared according to the process described in examples 1˜4 of WO2019052133.

In a particular embodiment, the non solvated crystalline form of daprodustat free acid is characterised by an X-ray powder diffraction (XRPD) pattern comprising at least five diffraction angles, when measured using Cu K_{α} radiation, selected from the group consisting of 4.0+/−0.2, 6.4+/−0.2, 7.5+/−0.2, 8.0+/−0.2, 15.2+/−0.2, 17.2+/−0.2, 18.6+/−0.2, 19.3+/−0.2, 19.9+/−0.2, 20.4+/−0.2, 21.0+/−0.2 and 24.1+/−0.2 degrees 2θ.

In a particular embodiment, the non solvated crystalline form of daprodustat free acid is characterised by an X-ray powder diffraction (XRPD) pattern comprising at least 6, 7, 8 or 9 diffraction angles, when measured using Cu K_{α} radiation, selected from the group consisting of 4.0+/−0.2, 6.4+/−0.2, 7.5+/−0.2, 8.0+/−0.2, 15.2+/−0.2, 17.2+/−0.2, 18.6+/−0.2, 19.3+/−0.2, 19.9+/−0.2, 20.4+/−0.2, 21.0+/−0.2 and 24.1+/−0.2 degrees 2θ.

In one embodiment, the non solvated crystalline form of daprodustat free acid is characterised by an X-ray powder diffraction (XRPD) pattern comprising at least the following diffraction angles: 6.4+/−0.2, 7.5+/−0.2 and 8.0+/−0.2 degrees 2θ.

In one embodiment, the non solvated crystalline form of daprodustat free acid is characterised by an X-ray powder diffraction (XRPD) pattern comprising at least the following diffraction angles: 6.4+/−0.2, 7.5+/−0.2, 8.0+/−0.2, 17.2+/−0.2 and 19.3+/−0.2 degrees 2θ.

In a more particular embodiment, the non solvated crystalline form of daprodustat free acid is characterised by an X-ray powder diffraction (XRPD) pattern comprising at least the following diffraction angles: 6.4+/−0.2, 7.5+/−0.2, 8.0+/−0.2, 15.2+/−0.2, 17.2+/−0.2 and 19.3+/−0.2 degrees 2θ.

In one embodiment, the non solvated crystalline form of daprodustat free acid is characterised by an X-ray powder diffraction (XRPD) pattern comprising characteristic XRPD peaks at 2theta values of 6.4°+/−0.2°, 7.5°+/−0.2°, 7.9°+/−0.2°. The X-ray powder diffraction pattern may show one or more additional characteristic peaks at 2theta values of 17.2°+/−0.2°, 21.0°+/−0.2°, 24.0°+/−0.2° or 19.3°+/−0.2°.

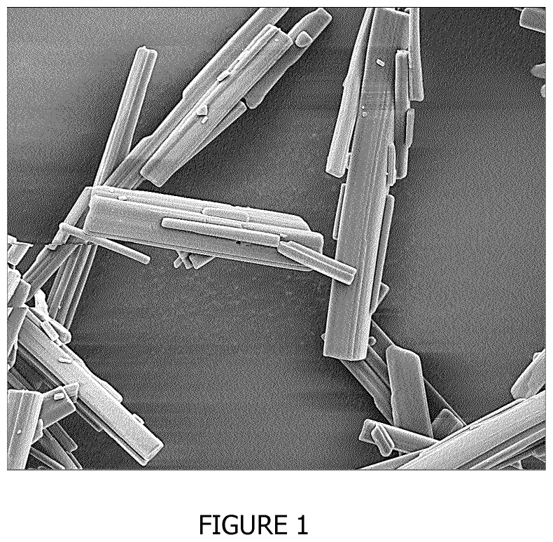

This non-solvated crystalline form of daprodustat free acid disclosed above has acicular crystals as shown in FIG. 1. Their shape results in the crystals having a low bulk density and poor flow as described in Example 1. The use of glidants to improve powder flow to facilitate formulation is well known in the art. As shown in Example 2, the use of glidants in direct admixture with the non-solvated crystalline form of daprodustat free acid resulted in tablets having a low tensile strength. Example 3 demonstrates that glidants can be omitted in compartments containing the non-solvated crystalline form of daprodustat free acid, and this results in tablets with good tensile strength and quality.

Tablet

In a first aspect, the invention provides an immediate release tablet comprising from 1 to 10 mg (measured as the free acid) daprodustat or a pharmaceutically acceptable salt thereof which has a tablet tensile strength of greater or equal to 1.7 MPa following compaction of the tablet core at a pressure in the range of 200 to 290 MPa. In more particular embodiments, the tablet tensile strength is greater than or equal to 1.75, 1.8, 1.9 or 2.0 MPa following compaction of the tablet core at a pressure in the range of 200 to 290 MPa. In particular embodiment, the immediate release tablet comprises from 1 to 8 mg (measured as the free acid) daprodustat or a pharmaceutically acceptable salt thereof. For the avoidance of doubt, the phrase “Main Compaction Pressure” used in Examples 5 and 6 refers to the pressure used for compaction of the tablet core.

In one embodiment, the immediate release tablet of the invention comprises a compartment containing daprodustat or a pharmaceutically acceptable salt thereof in an amount up to 5% based on the weight of the free acid, where the compartment does not contain a glidant. In one embodiment, the compartment contains the non solvated crystalline form of daprodustat free acid.

In one embodiment, the tablet is a monolithic tablet consisting of a single compartment of uniform composition that is optionally film coated. In one embodiment, the compartment is the tablet core. In another embodiment, the compartment is the entire tablet.

In an alternative embodiment, the tablet contains granules dispersed in an extragranular space and is optionally film coated. The granular and extragranular compositions may be different and form separate compartments. In one embodiment, the granular compartment is the compartment containing daprodustat or a pharmaceutically acceptable salt thereof (for example the non-solvated crystalline form of daprodustat free acid) and no glidant.

In one embodiment, the intragranular compartment comprises the crystalline form of non-solvated daprodustat free acid, a diluent, a binder and a disintegrant and no glidant. For the avoidance of doubt, more than one diluent, binder or disintegrant may be included. In one embodiment, the intragranular compartment consists of the crystalline form of non-solvated daprodustat free acid, one or more diluents, a binder and a disintegrant and no glidant.

In one embodiment, the extragranular compartment comprises a diluent, a disintegrant, a lubricant, and optionally a glidant. For the avoidance of doubt, more than one diluent, disintegrant, lubricant or glidant may be included. In one embodiment, the extragranular compartment consists of one or more diluents, a disintegrant, a lubricant, and optionally a glidant.

Suitable diluents include lactose, sucrose, dextrose, mannitol, sorbitol, starch (e.g. corn starch, potato starch, and pre-gelatinized starch), cellulose and its derivatives (e.g., microcrystalline cellulose), calcium sulfate, and dibasic calcium phosphate. In one embodiment, the diluent is not lactose.

Suitable binders include starch (e.g., corn starch, potato starch, and pre-gelatinized starch), hypromellose, gelatin, acacia, sodium alginate, alginic acid, tragacanth, guar gum, povidone, and cellulose and its derivatives (e.g. microcrystalline cellulose).

Suitable disintegrants include crospovidone, sodium starch glycolate, croscarmellose sodium, alginic acid, and sodium carboxymethyl cellulose.

Suitable lubricants include stearic acid, magnesium stearate, calcium stearate, and talc.

Glidants include colloidal silicon dioxide, talc, starch and magnesium stearate. In one embodiment, the glidant is colloidal silicon dioxide or magnesium stearate. In one embodiment, the glidant is silica. In another embodiment, the glidant is colloidal silicon dioxide.

In one embodiment, the invention provides an immediate release tablet, which tablet consists of:
a) intragranular components comprising the crystalline form of non-solvated daprodustat free acid, a diluent, a binder and a disintegrant; and
b) extragranular components comprising a diluent, a disintegrant, a lubricant, and optionally a glidant;
wherein the tablet is optionally coated.

In a more particular embodiment, the invention provides an immediate release tablet, which tablet consists of:
a) intragranular components consisting of the crystalline form of non-solvated daprodustat free acid and one or more diluents, one or more binders and one or more disintegrants; and
b) extragranular components comprising a diluent, a disintegrant, a lubricant, and optionally a glidant;
wherein the tablet is optionally coated.

A coating may be applied to the tablet core. An example of a commercially available coating is “OPADRY OY-S-28876 WHITE”. Coloured coatings are also commercially available.

In one embodiment, the immediate release tablet contains up to 76% by weight of intragranular components based on the weight of an uncoated tablet.

In one embodiment, the immediate release tablet comprises an intragranular compartment and an extragranular compartment wherein:
a. the intragranular components comprise:
   i. 1 to 10 mg of the crystalline form of non-solvated daprodustat free acid;
   ii. about 5 wt % hypromellose;
   iii. about 1.5 wt % croscarmellose sodium; and
   iv. mannitol and microcrystalline cellulose in a weight ratio from about 2.2 to about 3.6;
b. the extragranular components comprise, based on the total weight of the extragranular components:
   i. about 12 wt % croscarmellose sodium;
   ii. about 4 wt % magnesium stearate;
   iii. about 1.5% colloidal silica; and
   iv. mannitol and microcrystalline cellulose in a weight ratio of about 2.

In a particular embodiment, the tablet comprises about 1, 2 or 4 mg daprodustat and has a core tablet weight of about 150 mg. In another embodiment, the tablet comprises about 6, 8 or 10 mg daprodustat and has a core tablet weight of about 300 mg. In another embodiment, the tablet comprises about 6 or 8 mg daprodustat and has a core tablet weight of about 300 mg. The tablets described herein may be optionally film-coated.

In one embodiment, the immediate release tablet does not comprise lactose.

Medical Use

The immediate release tablet of the invention may be used in therapy, more particularly in the treatment of anemia. In a particular embodiment, the immediate release tablet of the invention may be used in the treatment of anemia due to chronic kidney disease (also known as renal anemia), anemia in patients with cancer receiving chemotherapy (including myelosuppressive or platinum containing chemotherapy), anemia in zidovudine-treated HIV-infected patients and anemia due to rheumatoid arthritis. In one embodiment, the immediate release tablet of the invention may be administered to patients receiving elective orthopaedic surgery.

Accordingly, in one embodiment, the invention provides the immediate release tablet of the invention for use in therapy.

In another embodiment, the invention provides the immediate release tablet of the invention for use in a method of treating anemia due to chronic kidney disease.

In yet another embodiment, the invention provides use of daprodustat or a pharmaceutically acceptable salt thereof in the manufacture of the immediate release tablet of the invention for use in the treatment of anemia due to chronic kidney disease.

In another embodiment, the invention provides a method for the treatment of anemia due to chronic kidney disease in a subject in need thereof, comprising administering to said subject the immediate release tablet of the invention.

Suitably, the subject is a mammal. In a particular embodiment, the subject is human.

In more particular embodiments, the subject having anemia due to chronic kidney disease may be receiving dialysis, for example haemodialysis or peritoneal dialysis. In another embodiment, the subject may be iron deficient (TSAT ≤20% and/or serum ferritin ≤100 ng/ml) and additionally receiving supplemental iron therapy.

In a further embodiment, the invention provides a dosage regimen for the treatment of anemia due to chronic kidney disease which aims to maintain haemoglobin in the range 10 to 12 g/dL and provide a safe increase in haemoglobin levels where haemoglobin levels are below this. The dose is modified based on the concentration of haemoglobin determined at clinical visits. Haemoglobin concentration may be measured by known methods for example HemoCue.

In one aspect, the invention provides a dosage regimen for the treatment of anemia due to chronic kidney disease for patients wherein the immediate release tablet of the invention is administered once daily at a dose of either 1 mg, 2 mg, 4 mg, 6 mg, 8 mg, 12 mg, 16 mg or 24 mg and wherein the dose is increased or decreased by one dose step based on the haemoglobin concentration of the patient to maintain the haemoglobin concentration of the patient within the range 10-12 g/dL. In one embodiment, the dose is increased or decreased by one dose step based on the haemoglobin concentration of the patient to maintain the haemoglobin concentration of the patient within the range 10-11 g/dL. In one embodiment, the dose is increased or decreased by one dose step based on the haemoglobin concentration of the patient to maintain the haemoglobin concentration of the patient at a target of 10 g/dL.

In particular embodiments, the haemoglobin concentration of the patient is monitored at least once every three months. In more particular embodiments, the haemoglobin concentration of the patient is monitored monthly or every four weeks. The skilled person will appreciate that monitoring may be more frequent when treatment is initiated, with the frequency of monitoring decreasing once the haemoglobin concentration of the patient has stabilised within the target range (10 to 12 g/dL or 10 to 11 g/dL or 10 g/dL).

In embodiments when there is a rapid increase in the haemoglobin concentration of the patient (e.g. exceeding 2.0 g/dL within 4 weeks), the dose is reduced by one dose step or interrupted.

In embodiments where the haemoglobin concentration of the patient exceeds the top end of the target range, the dose is interrupted until the haemoglobin concentration is in target range, and treatment is re-started at one dose level lower.

Clinical judgement is also important in dose increases and reductions. In embodiments where the patient is above the target range and at risk of thromboembolism (e.g. where a patient has had a stroke), the dose is reduced by one dose step or interrupted. In embodiments where the patient is exhibiting symptoms of anemia, the dose is increased by one dose step.

In one embodiment, the patient is not on dialysis. In another embodiment, the patient is on dialysis (e.g. haemodialysis or peritoneal dialysis).

In embodiments where the patient is not on dialysis and has not previously been treated with an erythropoiesis stimulating agent, the starting dose for the immediate release tablet of the invention is 4 mg once daily (where the patient has a haemoglobin concentration of <9.0 g/dL) or 2 mg once daily (where the patient has a haemoglobin concentration of ≥9.0 g/dL). In embodiments where the patient is not on dialysis and is being switched from an erythropoiesis stimulating agent, the starting dose for the immediate release tablet of the invention is 4 mg once daily. In embodiments where the patient is on dialysis, the starting dose for the immediate release tablet of the invention is 4 mg once daily.

In one aspect, the invention provides a dosage regimen for the treatment of anemia due to chronic kidney disease for patients on dialysis wherein the immediate release tablet of the invention is administered three times per week at a dose of either 2 mg, 4 mg, 8 mg, 12 mg, 16 mg, 24 mg, 32 mg or 48 mg and wherein the dose is increased or decreased by one dose step based on the haemoglobin concentration of the patient to maintain the haemoglobin concentration of the patient within the range 10-12 g/dL. In one embodiment, the dose is increased or decreased by one dose step based on the haemoglobin concentration of the patient to maintain the haemoglobin concentration of the patient within the range 10-11 g/dL.

In particular embodiments, the haemoglobin concentration of the patient is monitored at least once every three months. In more particular embodiments, the haemoglobin concentration of the patient is monitored monthly or every four weeks. The skilled person will appreciate that monitoring may be more frequent when treatment is initiated, with the frequency of monitoring decreasing once the haemoglobin concentration of the patient has stabilised within the target range (10 to 12 g/dL or 10 to 11 g/dL).

In embodiments when there is a rapid increase in the haemoglobin concentration of the patient (e.g. exceeding 2.0 g/dL within 4 weeks), the dose is reduced by one dose step or interrupted.

In embodiments where the haemoglobin concentration of the patient exceeds the top end of the target range, the dose is interrupted until the haemoglobin concentration is in target range, and treatment is re-started at one dose level lower.

Clinical judgement is also important in dose increases and reductions. In embodiments where the patient is above the target range and at risk of thromboembolism (e.g. where a patient has had a stroke), the dose is reduced by one dose step or interrupted. In embodiments where the patient is exhibiting symptoms of anemia, the dose is increased by one dose step.

In one embodiment, the starting dose for the immediate release tablet of the invention is 8, 12, 16 or 24 mg three times per week.

A dosage regimen for treatment of anemia due to chronic kidney disease to maintain haemoglobin concentration in the range 10-11 g/dL is provided, wherein the immediate release tablet of the invention is administered once daily at one of the following doses: 1, 2, 4, 6, 8, 10, 12, 16 and 24 mg (dose of free acid), and wherein:
a) where the haemoglobin concentration 12 g/dL, daprodustat therapy is ceased until the haemoglobin concentration <11.5 g/dL and therapy is commenced at one dose step lower;
b) where the haemoglobin concentration is in the range ≥9.5 to <11.5 g/dL, the dose is maintained;
c) where the haemoglobin concentration is in the range >11 to ≤11.5 g/dL at two consecutive clinic visits and there has been an increase or no change in the haemoglobin concentration since the last visit, the dose is reduced by one dose step;
d) where the haemoglobin concentration is in the range >11.5 to <12 g/dL and there has been a decrease in haemoglobin concentration since the last visit, the dose is maintained.
e) where the haemoglobin concentration is in the range >11.5 to <12 g/dL and there has been an increase or no change in the haemoglobin concentration since the last visit, the dose is reduced by one dose step;
f) where the haemoglobin concentration is in the range ≥9.5 to <10 at two consecutive clinic visits and there has been a decrease or no change in the haemoglobin concentration since the last visit, the dose is increased by one dose step;
g) where the haemoglobin concentration is in the range 7.5 to <9.5 g/dL and there has been an increase in haemoglobin concentration of ≥0.5 g/dL since the last visit, the dose is maintained;
h) where the haemoglobin concentration is in the range 7.5 to <9.5 g/dL and there has been a decrease, no change or an increase of <0.5 g/dL in haemoglobin concentration since the last visit, the dose is increased by one dose step;
i) where the haemoglobin concentration is <7.5 g/dL, the dose is increased by one dose step;
j) where there has been an increase in haemoglobin concentration of >2 g/dL over 4 weeks, or an increase in haemoglobin concentration of >1 g/dL over 2 weeks, the dose is reduced by one dose step; and
k) where there has been a decrease in haemoglobin concentration of >2 g/dL over 4 weeks, or a decrease in haemoglobin concentration of >1 g/dL over 2 weeks, the dose is increased by one dose step.

In one embodiment, the immediate release tablet of the invention is administered once daily at one of the following doses: 1, 2, 4, 6, 8, 12, 16 and 24 mg (dose of free acid) in accordance with the dosage regimen described herein.

In one embodiment, the invention provides the immediate release tablet of the invention for use in the treatment of anemia due to chronic kidney disease, wherein the immediate release tablet of the invention is administered once daily at one of the following doses: 1, 2, 4, 6, 8, 10, 12, 16 and 24 mg (dose of free acid) in accordance with a dosage regimen as described herein. In a more particular embodiment, the invention provides the immediate release tablet of the invention for use in the treatment of anemia due to chronic kidney disease, wherein the immediate release tablet of the invention is administered once daily at one of the following doses: 1, 2, 4, 6, 8, 12, 16 and 24 mg (dose of free acid) in accordance with a dosage regimen as described herein.

In one embodiment, the invention provides use of daprodustat or a pharmaceutically acceptable salt thereof in the manufacture of the immediate release tablet of the invention for use in the treatment of anemia due to chronic kidney disease, wherein the immediate release tablet of the invention is administered once daily at one of the following doses: 1, 2, 4, 6, 8, 10, 12, 16 and 24 mg (dose of free acid) in accordance with a dosage regimen as described herein. In a more particular embodiment, the invention provides use of daprodustat or a pharmaceutically acceptable salt thereof in the manufacture of the immediate release tablet of the invention for use in the treatment of anemia due to chronic kidney disease, wherein the immediate release tablet of the invention is administered once daily at one of the following doses: 1, 2, 4, 6, 8, 12, 16 and 24 mg (dose of free acid) in accordance with a dosage regimen as described herein.

In another aspect, the invention provides a dosage regimen for the treatment of anemia due to chronic kidney disease for patients on dialysis wherein the immediate release tablet of the invention is administered three times per week at a dose of either 2 mg, 4 mg, 8 mg, 12 mg, 16 mg, 24 mg, 32 mg or 48 mg (dose of free acid) and wherein the dose is increased or decreased by one dose step based on the haemoglobin concentration of the patient to maintain the haemoglobin concentration of the patient within the range 10-12 g/dL. In one embodiment, the dose is increased or decreased by one dose step based on the haemoglobin concentration of the patient to maintain the haemoglobin concentration of the patient within the range 10-11 g/dL.

In particular embodiments, the haemoglobin concentration of the patient is monitored at least once every three months. In more particular embodiments, the haemoglobin concentration of the patient is monitored monthly or every four weeks. The skilled person will appreciate that monitoring may be more frequent when treatment is initiated, with the frequency of monitoring decreasing once the haemoglobin concentration of the patient has stabilised within the target range (10 to 12 g/dL or 10 to 11 g/dL).

In embodiments when there is a rapid increase in the haemoglobin concentration of the patient (e.g. exceeding 2.0 g/dL within 4 weeks), the dose is reduced by one dose step or interrupted.

In one embodiment, the starting dose for the immediate release tablet of the invention is 8, 12, 16 or 24 mg three times per week.

A dosage regimen for treatment of anemia due to chronic kidney disease to maintain haemoglobin concentration in the range 10-11 g/dL is provided, wherein the immediate release tablet of the invention is administered three times per week at a dose of either 2 mg, 4 mg, 8 mg, 12 mg, 16 mg, 24 mg, 32 mg or 48 mg (dose of free acid), and wherein:
a) where the haemoglobin concentration ≥12 g/dL, daprodustat therapy is ceased until the haemoglobin concentration <11.5 g/dL and therapy is commenced at one dose step lower;
b) where the haemoglobin concentration is in the range ≥9.5 to <11.5 g/dL, the dose is maintained;
c) where the haemoglobin concentration is in the range >11 to ≤11.5 g/dL at two consecutive clinic visits and there has been an increase or no change in the haemoglobin concentration since the last visit, the dose is reduced by one dose step;
d) where the haemoglobin concentration is in the range >11.5 to <12 g/dL and there has been a decrease in haemoglobin concentration since the last visit, the dose is maintained.
e) where the haemoglobin concentration is in the range >11.5 to <12 g/dL and there has been an increase or no change in the haemoglobin concentration since the last visit, the dose is reduced by one dose step;
f) where the haemoglobin concentration is in the range ≥9.5 to <10 at two consecutive clinic visits and there has been a decrease or no change in the haemoglobin concentration since the last visit, the dose is increased by one dose step;
g) where the haemoglobin concentration is in the range 7.5 to <9.5 g/dL and there has been an increase in haemoglobin concentration of ≥0.5 g/dL since the last visit, the dose is maintained;
h) where the haemoglobin concentration is in the range 7.5 to <9.5 g/dL and there has been a decrease, no change or an increase of <0.5 g/dL in haemoglobin concentration since the last visit, the dose is increased by one dose step;
i) where the haemoglobin concentration is <7.5 g/dL, the dose is increased by one dose step;
j) where there has been an increase in haemoglobin concentration of >2 g/dL over 4 weeks, or an increase in haemoglobin concentration of >1 g/dL over 2 weeks, the dose is reduced by one dose step; and
k) where there has been a decrease in haemoglobin concentration of >2 g/dL over 4 weeks, or a decrease in haemoglobin concentration of >1 g/dL over 2 weeks, the dose is increased by one dose step.

In one embodiment, the invention provides the immediate release tablet of the invention for use in the treatment of anemia due to chronic kidney disease in patients on dialysis, wherein the immediate release tablet of the invention is administered three times per week at one of the following doses: 2 mg, 4 mg, 8 mg, 12 mg, 16 mg, 24 mg, 32 mg or 48 mg (dose of free acid) in accordance with a dosage regimen as described herein.

In one embodiment, the invention provides use of daprodustat or a pharmaceutically acceptable salt thereof in the manufacture of the immediate release tablet of the invention for use in the treatment of anemia due to chronic kidney disease in patients on dialysis, wherein the immediate release tablet of the invention is administered three times per week at one of the following doses: 2 mg, 4 mg, 8 mg, 12 mg, 16 mg, 24 mg, 32 mg or 48 mg (dose of free acid) in accordance with a dosage regimen as described herein.

For the avoidance of doubt, it is noted that any particular dose can be administered in a single tablet or multiple tablets. For example, the dose of 8 mg could be administered as a single 8 mg tablet, or two 4 mg tablets, or four 2 mg tablets or eight 1 mg tablets.

It will be apparent that dose adjustments will result in the daprodustat dose being increased or decreased by one dose step at a time. Those receiving the highest (maximum) dose of daprodustat who require a dose increase will maintain the same dose, while those receiving the lowest dose of daprodustat that require a dose decrease will finish daprodustat therapy.

### EXAMPLES

### Example 1: Characterisation of the Non-Solvated Crystalline Form of Daprodustat Free Acid

The non-solvated crystalline form of daprodustat free acid may be prepared as described in Examples 1-4 of WO2019052133 and shows characteristic XRPD peaks at 2theta values of 6.4°+/−0.2°, 7.5°+/−0.2°, 7.9°+/−0.2°. The X-ray powder diffraction pattern may show one or more additional characteristic peaks at 2theta values of 17.2°+/−0.2°, 21.0°+/−0.2°, 24.0°+/−0.2° or 19.3°+/−0.2°.

XRPD analysis of the non-solvated crystalline form of daprodustat free acid was conducted on a Philips X'Pert Pro Diffractometer, scanning the sample using the parameters listed in table 1.

TABLE 1

| Parameter: | Value: |
|---|---|
| Scan range | 2-40 degrees two-theta |
| Generator power | 40 kV, 40 mA |
| Radiation Source | Cu Kα |
| Scan type | Continuous |
| Time per step | 10 seconds |
| Step size | 0.017 degrees two-theta per step |
| Sample Rotation | 1 s revolution time |
| Incident Beam | 0.04 radian soller slits, 0.25 degree divergent slit, |
| optics | 10 mm beam mask, 0.5 degrees anti-scatter slit |
| Diffracted Beam | fixed slits (X'celerator module), 0.04 radian soller |
| optics | slits |
| Detector Type | Philips X'Celerator RTMS (Real Time Multi Strip) |

The sample was packed into a zero background silicon sample holder and gently flattened using a glass slide.

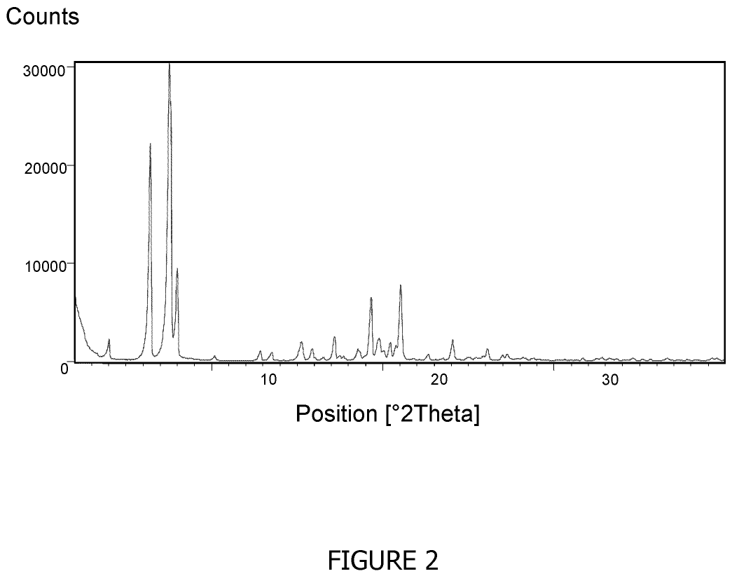

The XRPD pattern is shown in FIG. 2. Peaks identified in this pattern listed in table 2:

TABLE 2

| Diffraction angle (°2θ) | % Relative intensity |
|---|---|
| 6.3709 | 69.37 |
| 7.5742 | 100 |
| 7.958 | 21.64 |
| 12.8066 | 3.63 |
| 13.4622 | 3.48 |
| 15.2771 | 3.43 |
| 17.1618 | 3.59 |
| 18.5785 | 3.07 |
| 19.3046 | 18.81 |
| 19.8034 | 6.23 |
| 20.4071 | 3.95 |
| 21.0312 | 23.67 |
| 22.6261 | 1.88 |
| 24.0595 | 6.07 |
| 26.072 | 4.79 |
| 27.1063 | 1.29 |
| 39.3265 | 0.76 |
| 41.3744 | 2.52 |

Flowability of non-solvated crystalline form of daprodustat free acid was evaluated by calculating the Compressibility Index based on bulk and tapped density measurement of powders using USP <616> bulk and tapped density measurement method. Compressibility index of >25 indicates poor flowability, while <15 indicates good flowability.

Compressibility Index is calculated as 100×(1−Bulk density/Tapped density).

The Compressibility Index of non-solvated crystalline form of daprodustat free acid is >65, which indicates very poor flowability, with a very low bulk density (<0.2 g/mL).

### Comparative Example 2—Tablets with Glidant in the Daprodustat-Containing Compartment

Tablet formulations of the non-solvated crystalline form of daprodustat free acid containing a glidant in the granulation may be prepared as follows. The tablet cores comprise granules and extragranular components. Granules are prepared by adding daprodustat, mannitol, microcrystalline cellulose, hypromellose 2910, croscarmellose sodium and colloidal silicon dioxide into a high shear granulator. The powders are blended under high shear for at least 5 minutes and granulation performed while spraying at least 26% w/w purified water over a water addition time of at least 7 minutes and wet massing time of at least 2 minutes. The wet granules are dried in a fluid bed dryer to a target moisture content of not exceeding 2% w/w at a product temperature of at least 38° C. and the granules are dry milled to normalize granule size distribution. The milled granules are further blended with extragranular components mannitol, microcrystalline cellulose and croscarmellose sodium. Magnesium stearate is added and the resulting mixture is compressed using compaction pressures in the range 180 to 370 MPa into tablet cores using a rotary tablet press under the following conditions:

Tablet shape/size: round, biconvex tablets/7 mm diameter (≤4 mg dose); 9 mm diameter (≥6 mg dose)

Compression speed of at least 40000 tablets per hour.

The compositions of the tablets are provided in Table 3.

TABLE 3

| | Quantity (mg/tablet) | | | | |
|---|---|---|---|---|---|
| Component | 1 mg | 2 mg | 4 mg | 6 mg | 8 mg |

Granules

| | | | | | |
|---|---|---|---|---|---|
| Daprodustat | 1.00 | 2.00 | 4.00 | 6.00 | 8.00 |
| Mannitol | 71.74 | 71.05 | 69.66 | 140.71 | 139.32 |
| Microcrystalline Cellulose | 31.88 | 31.58 | 30.96 | 62.54 | 61.92 |
| Hypromellose 2910 | 5.63 | 5.63 | 5.63 | 11.25 | 11.25 |
| Croscarmellose Sodium | 1.69 | 1.69 | 1.69 | 3.38 | 3.38 |
| Colloidal Silicon Dioxide | 0.56 | 0.56 | 0.56 | 1.13 | 1.13 |
| Purified Water | — | — | — | — | — |

Extragranular Components

| | | | | | |
|---|---|---|---|---|---|
| Mannitol | 21.00 | 21.00 | 21.00 | 42.00 | 42.00 |
| Microcrystalline Cellulose | 10.50 | 10.50 | 10.50 | 21.00 | 21.00 |
| Croscarmellose Sodium | 4.50 | 4.50 | 4.50 | 9.00 | 9.00 |
| Magnesium Stearate | 1.50 | 1.50 | 1.50 | 3.00 | 3.00 |

| | | |
|---|---|---|
| Core tablet weight | 150.0 | 300.0 |

Purified water for granulation is removed during processing and does not remain in the tablet.

### Example 3—Tablets with Glidant Outside the Daprodustat-Containing Compartment

Tablet formulations of the non solvated crystalline form of daprodustat free acid containing a glidant in the extragranular matrix may be prepared as follows. The tablet cores comprise granules and extragranular components. Granules are prepared by adding daprodustat, mannitol, microcrystalline cellulose, hypromellose 2910 and croscarmellose sodium into a high shear granulator. The powders are blended under high shear for at least 5 minutes and granulation performed while spraying at least 26% w/w purified water over a water addition time of at least 7 minutes and wet massing time of at least 2 minutes. The wet granules are dried in a fluid bed dryer to a target moisture content of not exceeding 2% w/w at a product temperature of at least 38° C. and the granules are dry milled to normalize granule size distribution. The milled granules are further blended with extragranular components mannitol, microcrystalline cellulose, croscarmellose sodium and glidant colloidal silicon dioxide. Magnesium stearate is added and the resulting mixture is compressed using compaction pressures in the range 180 to 370 MPa into tablet cores using a rotary tablet press under the following conditions.

Tablet shape/size: round, biconvex tablets/7 mm diameter (≤4 mg); 9 mm diameter (≥6 mg)

Compression speed of at least 40000 tablets per hour

The compositions of the tablets are provided in Table 4.

TABLE 4

| | Quantity (mg/tablet) | | | | |
|---|---|---|---|---|---|
| Component | 1 mg | 2 mg | 4 mg | 6 mg | 8 mg |

Granules

| | | | | | |
|---|---|---|---|---|---|
| Daprodustat | 1.00 | 2.00 | 4.00 | 6.00 | 8.00 |
| Mannitol | 72.30 | 71.60 | 70.22 | 141.83 | 140.45 |
| Microcrystalline Cellulose | 31.88 | 31.58 | 30.96 | 62.54 | 61.92 |
| Hypromellose 2910 | 5.63 | 5.63 | 5.63 | 11.25 | 11.25 |
| Croscarmellose Sodium | 1.69 | 1.69 | 1.69 | 3.38 | 3.38 |
| Purified Water | — | — | — | — | — |

Extragranular Components

| | | | | | |
|---|---|---|---|---|---|
| Mannitol | 20.44 | 20.44 | 20.44 | 40.87 | 40.87 |
| Microcrystalline Cellulose | 10.50 | 10.50 | 10.50 | 21.00 | 21.00 |
| Croscarmellose Sodium | 4.50 | 4.50 | 4.50 | 9.00 | 9.00 |
| Colloidal Silicon Dioxide | 0.56 | 0.56 | 0.56 | 1.13 | 1.13 |
| Magnesium Stearate | 1.50 | 1.50 | 1.50 | 3.00 | 3.00 |

| | | |
|---|---|---|
| Core tablet weight | 150.0 | 300.0 |

Purified water for granulation is removed during processing and does not remain in the tablet.

### Example 4—Tablets without Glidant

Tablet formulations of the non solvated crystalline form of daprodustat free acid without glidant may be prepared as follows. The milled granules from Example 3 are blended with extragranular components mannitol, microcrystalline cellulose, croscarmellose sodium and magnesium stearate. Levels of extragranular mannitol and microcrystalline cellulose are adjusted while maintaining the same ratio to account for the removal of the glidant colloidal silicon dioxide to achieve the same target core tablet weights. The resulting mixture is compressed using compression pressures in the range 180 to 370 MPa into tablet cores using a rotary tablet press under the following conditions.

Tablet shape/size: round, biconvex tablets/7 mm diameter (≤4 mg); 9 mm diameter (≥6 mg)

Compression speed of at least 40000 tablets per hour

### Example 5—Measurement of Tablet Tensile Strength

Tablet tensile strength is measured on tablet cores using the method published by Pitt and Newton (Journal of Materials Science 23, 2723-2728, 1988). Results for the tablets of Example 2, 3 and 4 at a variety of compression pressures are shown in Table 5.

TABLE 5

| | Example 2 | | Example 3 | | | |
|---|---|---|---|---|---|---|
| | Intragranular glidant | | Extragranular glidant | | | |
| | | Tablet | | | Example 4 | |
| | | tensile | | Tablet | No glidant | |
| | Main | strength | Main | tensile | Main | Tablet tensile |
| | compaction | (MPa) | compaction | strength | compaction | strength |
| | pressure | Mean ± | pressure | (MPa) | pressure | (MPa) |
| Evaluation | (MPa) | SD | (MPa) | Mean ± SD | (MPa) | Mean ± SD |
| Tablet | 201.82 | 1.42 ± | 184.60 | 1.63 ± 0.08 | 186.02 | 1.38 ± 0.06 |
| tensile | | 0.04 | | | | |
| strength | 242.81 | 1.62 ± | 243.52 | 2.07 ± 0.02 | 243.95 | 1.60 ± 0.09 |
| (MPa) at | | 0.10 | | | | |
| 40000 | 291.06 | 1.80 ± | 299.17 | 2.29 ± 0.06 | 297.04 | 1.84 ± 0.09 |
| tablets per | | 0.08 | | | | |
| hour | 366.21 | 1.85 ± | 355.82 | 2.50 ± 0.10 | 357.10 | 1.76 ± 0.16 |
| compression | | 0.04 | | | | |
| speed | | | | | | |
| Tablet | 187.02 | 1.35 ± | 186.59 | 1.75 ± 0.08 | 188.02 | 1.39 ± 0.11 |
| tensile | | 0.07 | | | | |
| strength | 242.10 | 1.46 ± | 237.26 | 2.05 ± 0.10 | 243.52 | 1.71 ± 0.08 |
| (MPa) at | | 0.07 | | | | |
| 70000 | 297.04 | 1.78 ± | 305.58 | 2.39 ± 0.24 | 304.16 | 1.78 ± 0.12 |
| tablets per | | 0.07 | | | | |
| hour | 354.11 | 1.74 ± | 359.52 | 2.63 ± 0.07 | 351.27 | 1.74 ± 0.25 |
| compression | | 0.21 | | | | |
| speed | | | | | | |

### Example 6—Alternate Tablet Formulations without Glidant

Alternate tablet formulations containing 0.5 mg to 100 mg of the non-solvated crystalline form of daprodustat free acid without glidant may be prepared as follows. Granules are prepared by adding daprodustat, mannitol, microcrystalline cellulose, hypromellose 2910 and croscarmellose sodium into a high shear granulator. The powders are blended under high shear for at least 5 minutes and granulation performed while spraying at least 25% w/w purified water over a water addition time of at least 4 minutes and wet massing time of at least 1 minute. The wet granules are dried in a fluid bed dryer to a target moisture content and the granules are dry milled to normalize granule size distribution. The milled granules are further blended with extragranular components croscarmellose sodium, optionally mannitol and optionally microcrystalline cellulose. Magnesium stearate is added and the resulting mixture is compressed into tablet cores using a rotary tablet press to produce round, normal concave tablets 7.5 mm diameter (<5 mg) and 10 mm diameter (5 mg). The compositions of the tablets are provided in Table 6 and Table 7.

TABLE 6

| | Quantity (mg/tablet) | | | |
|---|---|---|---|---|
| Component | 2 mg | 5 mg | 25 mg | 100 mg |

Granules

| | | | | |
|---|---|---|---|---|
| Daprodustat, micronized | 2.00 | 5.00 | 25.00 | 100.00 |
| Mannitol | 47.00 | 117.50 | 36.58 | 146.31 |
| Microcrystalline Cellulose | 13.33 | 33.33 | 16.76 | 67.02 |
| Hypromellose 2910 | 3.33 | 8.33 | 4.19 | 16.76 |
| Croscarmellose Sodium | 1.00 | 2.50 | 1.26 | 5.03 |
| Purified Water | — | — | — | — |

Extragranular Components

| | | | | |
|---|---|---|---|---|
| Mannitol | 46.96 | 98.45 | 181.33 | NA |
| Microcrystalline Cellulose | 30.00 | 70.00 | 70.00 | NA |
| Croscarmellose Sodium | 4.50 | 10.50 | 10.50 | 10.50 |
| Magnesium Stearate | 1.88 | 4.38 | 4.38 | 4.38 |
| Core tablet weight | 150.0 | 350.0 | 350.0 | 350.0 |

Purified water for granulation is removed during processing and does not remain in the tablet.

TABLE 7

| | Quantity (mg/tablet) | | | | | |
|---|---|---|---|---|---|---|
| Component | 0.5 mg | 1 mg | 2 mg | 5 mg | 25 mg | 100 mg |

Granules

| | | | | | | |
|---|---|---|---|---|---|---|
| Daprodustat, | 0.50 | 1.00 | 2.00 | 5.00 | 25.00 | 100.00 |
| micronized | | | | | | |
| Mannitol | 11.75 | 23.50 | 47.00 | 117.50 | 29.78 | 119.09 |
| Microcrystalline | 3.33 | 6.67 | 13.33 | 33.33 | 15.01 | 60.03 |
| Cellulose | | | | | | |
| Hypromellose | 0.83 | 1.67 | 3.33 | 8.33 | 3.75 | 15.01 |
| 2910 | | | | | | |
| Croscarmellose | 0.25 | 0.50 | 1.00 | 2.50 | 1.50 | 6.00 |
| Sodium | | | | | | |
| Purified Water | — | — | — | — | — | — |

Extragranular Components

| | | | | | | |
|---|---|---|---|---|---|---|
| Mannitol | 96.95 | 80.29 | 46.96 | 98.45 | 190.08 | NA |
| Microcrystalline | 30.00 | 30.00 | 30.00 | 70.00 | 70.00 | 34.99 |
| Cellulose | | | | | | |
| Croscarmellose | 4.50 | 4.50 | 4.50 | 10.50 | 10.50 | 10.50 |
| Sodium | | | | | | |
| Magnesium | 1.88 | 1.88 | 1.88 | 4.38 | 4.38 | 4.38 |
| Stearate | | | | | | |
| Core tablet | 150.0 | 150.0 | 150.0 | 350.0 | 350.0 | 350.0 |
| weight | | | | | | |

Main compaction pressures in the range 156-224 MPa were used for the tablets in Table 7. Purified water for granulation is removed during processing and does not remain in the tablet.

Tablet tensile strength is measured on tablet cores using the method published by Pitt and Newton (Journal of Materials Science 23, 2723-2728, 1988). Tablet tensile strengths of these formulations range from 1.71-2.27 MPa.

What is claimed is:

1. An immediate release tablet comprising from 1 to 10 mg (measured as the free acid) daprodustat or a pharmaceutically acceptable salt thereof which has a tablet tensile strength of greater than or equal to 1.7 MPa following compaction of the tablet core at a pressure in the range of 200 to 290 MPa.

2. An immediate release tablet according to claim 1, wherein the tablet tensile strength is greater than or equal to 1.75 MPa following compaction of the tablet core at a pressure in the range of 200 to 290 MPa.

3. An immediate release tablet according to claim 1 wherein said daprodustat or a pharmaceutically acceptable salt thereof is a crystalline form of non-solvated daprodustat free acid characterised by an X-ray powder diffraction (XRPD) pattern comprising peaks at 6.4+/−0.2, 7.5+/−0.2 and 8.0+/−0.2 degrees 2θ.

4. An immediate release tablet according to claim 3, wherein the tablet comprises a compartment containing the crystalline form of non-solvated daprodustat free acid in an amount up to 5% based on the weight of the free acid, where the compartment does not contain a glidant.

5. An immediate release tablet according to claim 4, wherein the compartment is the entire tablet core.

6. An immediate release tablet according to claim 4, wherein the compartment is a granule within the tablet.

7. An immediate release tablet according to claim 6 which tablet consists of:
a) intragranular components comprising the crystalline form of non-solvated daprodustat free acid, a diluent, a binder and a disintegrant; and
b) extragranular components comprising a diluent, a disintegrant, a lubricant, and optionally a glidant;
wherein the tablet is optionally coated.

8. An immediate release tablet according to claim 7, which tablet consists of:
a) intragranular components consisting of the crystalline form of non-solvated daprodustat free acid and one or more diluents, one or more binders and one or more disintegrants; and
b) extragranular components comprising a diluent, a disintegrant, a lubricant, and optionally a glidant;
wherein the tablet is optionally coated.

9. An immediate release tablet according to claim 8, wherein the tablet contains up to 76% by weight of intragranular components based on the weight of the uncoated tablet.

10. An immediate release tablet according to claim 4, wherein the glidant is colloidal silicon dioxide.

11. An immediate release tablet according to claim 8, wherein:
i. the intragranular components comprise:
i. 1 to 10 mg of the crystalline form of non-solvated daprodustat free acid;
ii. about 5 wt % hypromellose;
iii. about 1.5 wt % croscarmellose sodium; and
iv mannitol and microcrystalline cellulose in a weight ratio from about 2.2 to about 3.6;
ii. the extragranular components comprise, based on the total weight of the extragranular components:
i. about 12 wt % croscarmellose sodium;
ii. about 4 wt % magnesium stearate;
iii. about 1.5% colloidal silica; and
iv. mannitol and microcrystalline cellulose in a weight ratio of about 2.

12. An immediate release tablet according to claim 11, wherein the tablet comprises about 1, 2 or 4 mg daprodustat and has a core tablet weight of about 150 mg.

13. An immediate release tablet according to claim 11, wherein the tablet comprises about 6 or 8 mg daprodustat and has a core tablet weight of about 300 mg.

14. An immediate release tablet according to claim 12, wherein the tablet is film-coated.

15. An immediate release tablet according to claim 13, wherein the tablet does not comprise lactose.

16-27. (canceled)

28. A method for the treatment of anemia due to chronic kidney disease in a subject in need thereof, comprising administering to said subject the immediate release tablet according to claim 1.

29. A method according to claim 28, wherein the subject is receiving dialysis.

30. A method according to claim 28, wherein the subject is iron deficient and wherein the subject additionally receives supplemental iron therapy.

31. A method according to claim 28, wherein the immediate release tablet of the invention is administered once daily at a dose of either 1 mg, 2 mg, 4 mg, 6 mg, 8 mg, 12 mg, 18 mg or 24 mg and wherein the dose is increased or decreased by one dose step based on the haemoglobin concentration of the patient to maintain the haemoglobin concentration of the patient within the range 10-11 g/dL.

32. A method according to claim 31, wherein the patient is not on dialysis.

33. A method according to claim 32 wherein the starting dose for the immediate release tablet is 4 mg once daily (where the patient has previously been treated with an erythropoiesis stimulating agent, or where the patient has not previously been treated with an erythropoiesis stimulating agent and has a haemoglobin concentration of <9/0 g/dL) or 2 mg once daily (where the patient has not previously been treated with an erythropoiesis stimulating agent and has a haemoglobin concentration of ≥9.0 g/dL).

34. A method according to claim 28, wherein the immediate release tablet is administered three times per week at a dose of either 2 mg, 4 mg, 8 mg, 12 mg, 16 mg, 24 mg, 36 mg or 48 mg and wherein the dose is increased or decreased by one dose step based on the haemoglobin concentration of the patient to maintain the haemoglobin concentration of the patient within the range 10-11 g/dL.

35. A method according to claim 34, wherein the patient is on dialysis.

36. A method according to claim 35, wherein the starting dose is 8, 12, 16 or 24 mg three times per week.

37. A method according to claim 31, wherein the haemoglobin concentration of the patient is monitored at least once every three months.

38. A method according to claim 34, wherein the haemoglobin concentration of the patient is monitored at least once every three months.

39. A method according to claim 37, wherein when there is an increase in haemoglobin concentration of the patient exceeding 2.0 g/dL within 4 weeks, the dose is reduced by one dose step or interrupted.

40. A method according to claim 38, wherein when there is an increase in haemoglobin concentration of the patient exceeding 2.0 g/dL within 4 weeks, the dose is reduced by one dose step or interrupted.
